(19) United States
(12) Patent Application Publication
(10) Pub. No.: US 2024/0058344 A1
(43) Pub. Date: Feb. 22, 2024

(54) METHODS OF TREATING NEURODEGENERATIVE DISORDERS AND STAT3-LINKED CANCERS USING SUPPRESSORS OF ELECTRON LEAK

(71) Applicant: Cornell University, lthaca, NY (US)

(72) Inventors: Adam Orr, Briarcliff Manor, NY (US); Anna Orr, Briarcliff Manor, NY (US)

(21) Appl. No.: 18/268,102

(22) PCT Filed: Dec. 17, 2021
(86) PCT No.: PCT/US21/64070

§ 371 Date: Jun. 16, 2023

Related U.S. Application Data
(60) Provisional application No. 63/127,624, filed on Dec. 18, 2020.

(51) Int. Cl. A61K 31/519; A61K 31/18; A61K 31/343; A61K 31/385; A61K 31/426; A61K 31/498; A61K 31/517; A61P 25/28; A61P 35/00

(52) U.S. Cl. CPC A61K 31/519; A61K 31/18; A61K 31/343; A61K 31/385; A61K 31/426; A61K 31/498; A61K 31/517; A61P 25/28; A61P 35/00

(57) ABSTRACT

Disclosed herein are methods of treating or preventing neurodegenerative disease, neuronal damage, neuroinflammation, or cancer using suppressors of electron leak.

### RELATED APPLICIATIONS

This application claims the benefit of U.S. Provisional Application No. 63/127,624, filed Dec. 18, 2020, the contents of which are fully incorporated by reference herein.

### GOVERNMENT SUPPORT

This invention was made with government support under Grant Numbers 1R01AG068091-01, awarded by National Institutes of Health. The government has certain rights in the invention.

### BACKGROUND

Despite a growing understanding of the various pathogenic mechanisms contributing to neurodegeneration, there are currently no disease-modifying treatments available for Frontotemporal Dementia (FTD) or related dementias. Treatment strategies based on new targets are essential for realizing this critical need.

### SUMMARY OF THE INVENTION

The site-specific inhibitors of mitochondrial reactive oxygen species (ROS) production, or Suppressors of Electron Leak (SELs), offer a promising approach to the treatment of FTD and other dementias due to SELs' unique mechanism of action and disease-modifying properties.

In certain aspects, the present disclosure provides methods of treating or preventing a neurodegenerative disease or neuronal damage, comprising administering a therapeutically effective amount of a S1QEL or a S3QEL to a subject in need thereof.

In certain aspects, the present disclosure provides methods of reducing neuroinflammation or glial alteration in the brain of a subject, comprising administering a therapeutically effective amount of a S1QEL or a S3QEL to a subject in need thereof.

In certain aspects, the present disclosure provides methods of treating cancer in a subject, comprising administering a therapeutically effective amount of a S1QEL or a S3QEL to a subject in need thereof.

### BRIEF DESCRIPTION OF THE DRAWINGS

FIGS. 1A-F show that S3QEL2 and S1QEL1.1 cross the blood-brain barrier and are well-tolerated during chronic administration. (FIGS. 1A-B) Levels of S3QEL2 (FIG. 1A) and S1QEL1.1 (FIG. 1B) in the brain following a single i.p. injection or oral consumption (per oral, PO), which improved duration of exposure. n=3-6 wild-type mice per group. (FIGS. 1C-D) Body weights of male nontransgenic (NTG) or hTauP301S mice treated with S3QEL2 (FIG. 1C) (S3, 5 mg/kg/day, PO in almond butter), S1QEL1.1 (FIG. 1D) (10 mg/kg/day, PO in almond butter), or vehicle. (FIG. 1E) Levels of S3QEL2 in the mouse brain following almond butter (AB) or chow administration at indicated doses for 21 days. (n=15 mice per dose). n=3-15 wild-type mice per condition. (FIG. 1F) Body weight of 4-5-month-old wild-type male mice treated with formulated chow containing S3QEL2 at indicated doses and durations (n=15 mice per dose).

FIGS. 2A-B show that S3QEL2 modulated oxidative pathways in hTauP301S mice. (FIG. 2A) RNA levels of neuroinflammatory markers in 8-month-old male nontransgenic (NTG) and hTauP301S mice. n=3 mice per group. (FIG. 2B) Hippocampal RNA levels of Nrf2 in 10-month-old mice treated with S3QEL2 (S3, 5 mg/kg/day) or vehicle. n=3-6 mice per group. *p<0.05; **p<0.01; ANOVA with Dunnett's test.

FIG. 3 shows that S3QEL2 and S1QEL1.1 reduced gene expression linked to glial reactivity and neuroinflammation in hTauP301S mice. Subset of neuroinflammatory markers measured by RT-qPCR in hippocampi of 10-month-old male nontransgenic (NTG) or hTauP301S mice after 6 weeks of oral dosing with 5 mg/kg/day S3QEL2, 10 mg/kg/day S1QEL1.1, or vehicle. Most of the upregulated genes were reduced by S1QEL1.1 or S3QEL2. Average fold-change vs NTG. n=3-5 mice per group.

FIGS. 4A-E shows that S3QEL2 reduced hippocampal astrogliosis and phosphorylated tau levels in hTauP301S mice. Immunofluorescent staining and quantification of astrocytic marker GFAP (red, FIG. 4A) and phospho-serine 202/205 tau (AT8, green, FIG. 4B) in male nontransgenic (NTG) and hTauP301S mice after 6 weeks of oral dosing with S3QEL2 or vehicle. (FIGS. 4C-4E) n=12-16 mice per group; *p<0.05, **p<0.01, ***p<0.001, ANOVA with Bonferroni's test.

FIGS. 5A-B shows that S3QEL2 reduced phosphorylated, but not total tau protein, in hTauP301S mice. Levels of phospho-tau (FIG. 5A) and total tau (FIG. 5B) as measured by Western blotting in hippocampal tissue from male nontransgenic (NTG) and hTauP301S mice after 6 weeks of oral dosing with S3QEL2 or vehicle. n=3-6 mice per group. *p<0.05, **p<0.01, ***p<0.001, ANOVA with Dunnett's test.

FIGS. 6A-B shows that S3QEL2 reduced protein markers of neuroinflammation in hTauP301S mice. Levels of immunolabeling for the inflammasome protein ASC/Pycard (FIG. 6A) and the microglial marker CD11b (FIG. 6B) in hippocampal tissue from nontransgenic (NTG) and hTauP301S mice after 6 weeks of oral dosing with S3QEL2 or vehicle. n=3-6 mice per group. *p<0.05, **p<0.01, ***p<0.001, ANOVA with Dunnett's test.

FIG. 7 shows that S3QEL2 reduced early mortality in hTauP301S mice. Nontransgenic (NTG) and transgenic hTauP301S male mice received oral dosing with S3QEL2-formulated chow or control chow (Control) for indicated durations starting at 4-5 months of age. n=14-15 mice per genotype and treatment, pairwise Mantel-Cox test.

FIGS. 8A-D shows that Tau dysfunction induces mitochondrial oxidative stress and dendritic loss, and antioxidants suppress tau-mediated damage in neurons. (FIG. 8A) Primary neurons immunostained for human tau after transduction with AAVs encoding human wild-type (WT) or P301S mutant tau under the synapsin-1 promoter. (FIGS. 8B-8C) Non-reducing blots of oxidized mitochondria-specific H₂O₂-detoxifying enzyme peroxiredoxin-3 (PRDX3, dimer). (FIG. 8D) Omitting antioxidants from media exacerbates the effects of tau overexpression. n=3 per condition; *p<0.05 vs. +Antiox; ANOVA with Bonferroni's test.

FIGS. 9A-D shows that S3QEL2 did not affect tau-associated neuronal damage in primary neurons cultured alone or with microglia. Primary neurons were immunostained for MAP2 or NeuN following mock treatment or transduction with AAV encoding human P301S mutant tau under the synapsin-1 promoter. Neurons were cultured without (FIGS. 9A-9B) or with (FIGS. 9C-9D) primary mouse microglia. S3QEL2 (S3, 10 μM). n=4 per condition.

FIGS. 10A-C shows that S3QEL2 prevented neuronal damage and aberrant increases in neuronal firing in astrocytic-neuronal co-cultures. (FIG. 10A) Co-cultures were immunostained for NeuN following transduction with AAV encoding human P301S mutant tau. Vehicle (Veh) or S3QEL2 (S3, 1μM) was added on day 8 in vitro and cells were analyzed on day 14. (FIG. 10B) Example traces recorded using multi-electrode array (MEA). Spikes (black) and network bursts (pink boxes). Top traces show population activities. (FIG. 10C) Average neuronal firing (% of baseline) in co-cultures. Mock treatment (control) or AAV-TauP301S with or without S3QEL2 (S3, 1μM) were added on day 6. n=4 wells per condition, *p<0.05, ANOVA with Bonferroni's test.

FIGS. 11A-B shows that S3QEL2 reduced markers of reactivity and immune-linked signaling in primary astrocytes. (FIGS. 11A-11B) Astrocytes isolated from P2-P3 mice were treated with oligomeric Aβ (3 μM) and S3QEL2 (S3, 20 μM) or vehicle (V) for 24 h and analyzed by qRT-PCR (FIG. 11A) or Western blotting (B) and normalized to vehicle controls. (FIG. 11B) Phospho-STAT3 levels were normalized to total STAT3 per sample. n=3 wells per condition; *p<0.05, **p<0.01, ***p<0.001, ANOVA with Dunnett's test.

FIGS. 12A-B shows that S3QEL2 reduced phospho-STAT3 and total STAT3 levels in primary astrocytes. (FIGS. 12A-12B) Astrocytes isolated from P2-P3 mice were treated with oligomeric Aβ (3 μM) (A) or A1 cocktail consisting of 30 ng/mL TNF-α, 3 ng/mL IL-1α, and 400 ng/mL Clq (B), and S3QEL2 (S3, 60 μM) or vehicle (Con) for indicated durations and analyzed by Western blotting. Phospho-STAT3 levels were normalized to total STAT3 per sample, and total STAT3 levels were normalized to γ-tubulin levels per sample. n≥3 wells per condition; *p<0.05, **p<0.01, ***p<0.001, one-way ANOVA with Tukey's multiple comparisons test.

FIG. 13 shows that S3QEL2 reduced the levels of lipocalin-2 protein released by primary astrocytes. Astrocytes isolated from P2-P3 mice were treated with A1 cocktail consisting of 30 ng/mL TNF-α, 3 ng/mL IL-1α, and 400 ng/mL Clq, and S3QEL2 (S3, 60 μM) or vehicle (Con) for approximately 24 h. Levels of astrocytic lipocalin-2 protein released into the culture media were analyzed by ELISA. n=4 wells per condition. **p<0.01, ***p<0.001, one-way ANOVA with Dunnett's multiple comparisons test.

FIG. 14 shows that S3QEL2 did not affect markers of reactivity and immune signaling in isolated microglia. Microglia isolated from P2-P3 mice were treated with lipopolysaccharide (LPS, 100 ng/ml) or oligomeric Aβ (3 μM), and S3QEL2 (20 μM) or vehicle for 24 h and analyzed by qRT-PCR. n=3 wells per condition.

FIGS. 15A-C shows that S3QEL2 dose-dependently inhibited cell proliferation in two different glioblastoma cell lines, including A-172 (FIG. 15A) and T98G (FIG. 15B), but had minimal effect on U-87 MG line (FIG. 15C) 72 and 96 h after S3QEL2 application. Relative cell density was measured using the CellTiter-Glo luciferase assay. n=3 wells per condition. *p<0.05, **p<0.01, ***p<0.001 vs. 0 μM, one-way ANOVA with Dunnett's multiple comparisons test.

FIGS. 16A-C shows that S3QEL2 inhibited cell proliferation of the glioblastoma cell line A-172 (FIG. 16A), but not U-87 MG line (FIG. 16B) at 72 h after S3QEL application. Cell density was measured using DAPI staining of cell nuclei and quantification of fluorescence intensities. n=3 wells per condition. FIG. 16C shows relative STAT3 protein levels in the different glioblastoma lines (A-172, T98G, and U-87 MG cells). STAT3 levels were measured by Western blotting. STAT3 levels were normalized to γ-tubulin levels within each cell lysate and expressed as fold change relative to U-87MG cells. n=4 wells per condition. **p<0.01, ***p<0.001 vs. 0 μM or U-87MG, one-way ANOVA with Dunnett's multiple comparisons test.

FIGS. 17A-C shows that different S3QEL analogs, S3QEL1.2 and S3QEL2, can reduce phospho-STAT3 and H₂O₂ production in primary astrocytes. (FIG. 17A) Astrocytes isolated from P2-P3 mice were treated with A1 cocktail consisting of 30 ng/ml TNF-α, 3 ng/ml IL-1α, and 400 ng/ml Clq, and S3QEL1.2 (3 μM) or vehicle for approximately 6 h and analyzed by Western blotting. Phospho-STAT3 levels were normalized to total STAT3 per sample. n=6 wells per condition. **p<0.01, ***p<0.001, one-way ANOVA with Dunnett's multiple comparisons test. (FIG. 17B) Astrocytes isolated from P2-P3 mice were treated with A1 cocktail consisting of 30 ng/ml TNF-α, 3 ng/ml IL-1α, and 400 ng/ml Clq for 24 h, and analyzed for levels of released H₂O₂ using Amplex UltraRed, a fluorogenic substrate that is a highly sensitive indicator of H₂O₂, in the presence of horseradish peroxidase and superoxide dismutase. n=6 wells per condition. *p=0.01, Student's t test. (FIG. 17C) Astrocytes isolated from P2-P3 mice were treated with vehicle or 3 ng/ml IL-1α and either S3QEL2 (20 μM), S3QEL1.2 (1 μM), or vehicle for approximately 24 h, and analyzed for levels of released H₂O₂ using Amplex UltraRed. n=6 wells per condition. *p<0.05, **p<0.01, ***p<0.001, two-way ANOVA with Bonferroni's multiple comparisons test.

FIG. 18 shows that S3QEL2 and S3QEL1.2 do not affect intracellular ATP levels in primary astrocytes at the indicated concentrations, consistent with previous studies that these compounds are selective for complex III ROS production and do not affect other mitochondrial functions such as ATP production. Antimycin A and myxothiazol were used as positive controls to inhibit mitochondrial respiration and ATP production. n=3 wells per condition. ***p<0.001 vs. vehicle, mixed-effects model with Dunnett's multiple comparisons test.

FIG. 19 shows that S3QEL2 does not affect mitochondrial respiration in astrocytes. Oxygen consumption rates (OCR) and maximal consumption rates are shown for indicated S3QEL2 concentrations and time points. Antimycin and rotenone were used as positive controls to inhibit mitochondrial respiration. ***p<0.001 vs. vehicle, two-way repeated measures ANOVA with Dunnett's multiple comparisons test.

### DETAILED DESCRIPTION OF THE INVENTION

Tau and TDP-43 Pathology in FTD and Related Dementias. Two prevalent hallmarks of FTD neuropathology are the formation of intracellular neurofibrillary tangles containing the microtubule-binding protein tau and the dysregulation of the DNA/RNA-binding protein TDP-43. Direct effects of aberrant tau on multiple cellular components, including microtubules and mitochondria, likely cause clinical decline and neurodegeneration. Indeed, tangles correlate with cognitive decline, and are prevalent in neurodegenerative conditions. Several therapeutic strategies for reducing tau pathology are being explored, but their clinical safety and efficacy remain unproven. Thus, there is not yet a definitive strategy for reducing tau-related pathogenesis. TDP-43 is a multifunctional and ubiquitous DNA/RNA-binding protein, and is a major component of protein inclusions irn FTD and ALS. Under normal conditions, TDP-43 is enriched in the nucleus and involved in regulating RNA transcription, splicing, and transport, among other processes. It is not yet clear how TDP-43 dysfunction promotes impairments in disease (i.e. whether impairments are caused by loss of function, gain of toxic function, or both). Given its importance for gene expression and other functions, TDP-43 and its direct effectors may not be optimal therapeutic targets.

Mitochondrial ROS in FTD and Related Dementias. Mitochondrial dysfunction and ROS production are increasingly recognized as key factors in neurodegenerative disease. Mitochondria couple metabolism and respiration to energy production. However, these processes can also generate high levels of ROS via direct leak of electrons to oxygen. Even in healthy cells, mitochondria are major contributors to total ROS levels, and mitochondrial impairments can dramatically increase ROS production. Notably, mitochondrial dysfunction and oxidative stress are prevalent features of FTD and other dementias, and mitochondrial ROS are implicated as central, feed-forward drivers of cellular disruption in dementia, including impaired calcium buffering, protein misfolding, and neuroinflammation. Recent studies show that the major contributing factors in FTD, including aging, abnormal tau accumulation, TDP-43 dysregulation, and C9ORF72 hexanucleotide expansion, are causally linked to mitochondrial dysfunction and increases in mitochondrial ROS. Several FTD-linked genes, including VCP and TBK1, are also involved in mitochondrial function. Notably, tau dysfunction impairs mitochondria and increases oxidative stress, and mitochondrial impairments can, in turn, influence tau phosphorylation and cell homeostasis. Likewise, TDP-43 dysfunction impairs mitochondrial homeostasis and increases ROS, possibly via dysregulation of mitochondrial gene transcription and expression of complex I. In addition, dipeptide repeat protein accumulation linked to C9ORF72 causes oxidative stress and mitochondrial deficits that promote DNA damage. Similarly, amyloid-β production and neurotoxicity may be dependent on mitochondrial ROS. Alterations in mitochondria and ROS are also linked to aging.

Tauopathy and other FTD-linked proteinopathies trigger immune signaling and neuroinflammation, which are dependent on mitochondrial ROS. Indeed, mitochondrial ROS may be essential activators of inflammasomes, which may promote neuroinflammation and aberrant glial responses in dementia. Genetic knockout or depletion of key mediators of inflammasome activation prevents cognitive deficits and neuropathology in mouse models. In addition to inflammasomes, mitochondrial ROS promote several other immune-related pathways implicated in disease, including NF-κB, ERK1/2, and JAK/STAT signaling.

Emerging Roles of Complex III ROS in Disease. Mitochondria generate ROS from at least 11 distinct sites, and respiratory complexes I and III are considered to be the major sources. Although the roles of each site are not completely understood, complex III is the highest capacity site of mitochondrial ROS production due in part to its ubiquitous and high expression. Collectively, maximal rate of complex III ROS is often twice the maximal rates of complex I and II in mitochondria from diverse tissues. Complex III is also poised to affect intracellular signaling due to its ability to generate ROS toward the cytosol via ROS diffusion from the intetmembrane space, whereas complex I produces ROS exclusively towards the internal mitochondrial matrix where ROS are detoxified by mitochondrial antioxidant systems. The first reported role of complex III ROS was hypoxic stabilization of HIF-1α, a role recently confirmed using suppressors of complex III site Q electron leak, S3QELs (pronounced “Sequels”). Complex III ROS is also essential for inflammatory signaling. Specifically, complex III ROS mediates ERK1/2 and NF-κB activation and cytokine secretion via disulfide oxidation of the IKK regulator NEMO. It may also promote inflammasome activation. Genetic manipulations suggest complex III ROS promotes T cell activation and antigen-specific expansion. S3QELs have been used to further establish the roles of complex III ROS in toll-like receptor signaling, immunological synapse formation, and cytokine production. In addition to inflammatory cascades, complex III ROS is increasingly implicated in pathways related to aging and dementia, including programmed cell death, hypoxia-induced HIF-1α, and ER stress. The data presented in the present disclosure further suggest that complex III ROS is involved in tauopathy and glial reactivity in disease.

Therapeutic Thrgeting of Mitochondrial ROS. Given the intimate link between mitochondrial energy and ROS production, the path toward therapeutics that target ROS without unwanted side effects on energy levels and metabolism has not been straightforward. Current genetic and pharmacologic tools to block mitochondrial ROS invariably affect metabolism and disrupt cellular redox, and therefore have limited clinical applications. Antioxidants, which remove ROS after production, are not selective for specific sites of ROS production and often have poor selectivity for specific types of ROS (e.g., superoxide vs hydrogen peroxide). Also, possibly due to low selectivity, antioxidants typically require high doses and have poor efficacy in the clinic. They can also affect redox homeostasis and metabolism, and display pro-oxidant activity and toxicity.

Newly discovered SELs, including S1QELs and S3QELs, are innovative due to their ability to block ROS production from a single site in the mitochondria without altering normal functions like ATP production or inducing various other mitochondrial and cellular off-target effects. These SELs are potent (IC₅₀ in nM to low μM) and efficacious in diverse systems, including cultured cells and Drosophila. The present disclosure shows that using SELs to target a specific site of mitochondrial ROS production can alleviate tau pathology in cell and animal models, possibly via modulation of immune-related responses in glial cells.

Importantly, if kept below saturating concentrations, effective doses of S1QELs and S3QELs have not shown signs of toxicity in cells or mice with extended treatments (days to months). In a wide array of mitochondrial and cellular assays, signs of off-target activities were not observed except subtle membrane depolarization of isolated mitochondria at very high doses (>20-fold IC50), though no signs of depolarization in intact cells. Based on expected physiological roles of complex III ROS, chronic and systemic exposure to S1QELs and S3QELs might alter differentiation of certain stem cells, hypoxia/oxygen sensing, and immune cell activation.

The studies in wild-type mice (>10 mo treatment duration) have shown no adverse events. In contrast, in addition to the expected protection from dementia-linked proteinopathy, it is predicted that SELs might more broadly (1) suppress central and peripheral immune hyperactivation, (2) inhibit tumorigenesis and general aging processes, and (3) rebalance redox systems, thereby affecting diverse neurodegenerative cascades associated with FTD and other dementias. Therefore, SELs are prime leads for therapeutic development.

### Methods of Use

In certain aspects, the present disclosure provides methods of treating or preventing a neurodegenerative disease or neuronal damage, comprising administering a therapeutically effective amount of a S1QEL or a S3QEL to a subject in need thereof.

In certain embodiments, following administration of the S1QEL or S3QEL, an inflammatory marker or a glial reactivity marker in the subject's brain is reduced.

In certain embodiments, the neurodegenerative disease is a tauopathy. In certain embodiments, the tauopathy is dementia, Alzheimer's disease, Parkinson's disease, progressive supranuclear palsy, corticobasal degeneration, argyrophilic grain disease, or chronic traumatic encephalopathy. In certain embodiments, the tauopathy is dementia, Alzheimer's disease, or Parkinson's disease. In certain embodiments, the neurodegenerative disease is Alzheimer's disease. In certain embodiments, the neurodegenerative disease is Parkinson's disease. In certain preferred embodiments, the neurodegenerative disease is dementia, such as frontotemporal dementia (FTD) or amyotrophic lateral sclerosis. In certain preferred embodiments, the neurodegenerative disease is dementia, such as frontotemporal dementia (FTD).

In certain embodiments, the neurodegenerative disease or neuronal damage is related to A1/Reactive Astrocyte Involvement. In certain embodiments, the neurodegenerative disease or neuronal damage related to A1/Reactive Astrocyte Involvement is Alzheimer's disease, Parkinson's disease, cerebral amyloid angiopathy, chronic pain, Creutzfeldt-Jakob disease, depression, Huntington's disease, spinal cord injury, or traumatic brain injury.

In certain embodiments, the neurodegenerative disease or neuronal damage is related to IL-1a-b. In certain embodiments, the neurodegenerative disease or neuronal damage related to IL-1a-b is Alzheimer's disease, Parkinson's disease, HIV-associated neurodegeneration, depression, amyotrophic lateral sclerosis, frontotemporal dementia, chronic pain, intracerebral hemorrhage, multiple sclerosis, stroke, traumatic brain injury, vascular dementia, Huntington's disease, or spinal cord injury.

In certain embodiments, the neurodegenerative disease or neuronal damage is related to Lipocalin-2. In certain embodiments, the neurodegenerative disease or neuronal damage related to Lipocalin-2 is Alzheimer's disease, Parkinson's disease, HIV-associated neurodegeneration, depression, amyotrophic lateral sclerosis, frontotemporal dementia, chronic pain, intracerebral hemorrhage, multiple sclerosis, stroke, traumatic brain injury, or vascular dementia.

In certain embodiments, the neurodegenerative disease or neuronal damage is related to STAT3. In certain embodiments, the neurodegenerative disease or neuronal damage related to STAT3 is Alzheimer's disease, Parkinson's disease, cerebral amyloid angiopathy, chronic pain, multiple sclerosis, spinal cord injury, medullary thyroid carcinoma, or glioblastoma multiforme.

In certain embodiments, the tissue or cells exhibiting neurodegenerative disease or neuronal damage have aberrantly increased STAT3 levels. For example, the tissue or cells exhibiting neurodegenerative disease or neuronal damage may have at least about 1.1, at least about 1.2, at least about 1.3, at least about 1.4, or at least about 1.5 fold increase in total STAT3 relative to those tissue or cells without neurodegenerative disease or neuronal damage. In some embodiments, the increase in STAT3 is relative to those without neurodegenerative disease or neuronal damage who are not responsive to a S1QEL and/or a S3QEL. In exemplary embodiments, the tissue or cells exhibiting neurodegenerative disease or neuronal damage having sensitivity to an S3QEL had an increased total STAT3 as compared to those not responsive to S3QEL.

In certain embodiments, the tissue or cells exhibiting neurodegenerative disease or neuronal damage have aberrantly active STAT3. For example, the tissue or cells exhibiting the neurodegenerative disease or neuronal damage may have at least about 1.1, at least about 1.2, at least about 1.3, at least about 1.4, or at least about 1.5 fold increase in active STAT3 relative to those tissue or cells without the neurodegenerative disease or neuronal damage. In some embodiments, the increase in active STAT3 is relative to those without neurodegenerative disease or neuronal damage who are not responsive to a S1QEL and/or a S3QEL. In some embodiments, the increase in active STAT3 is relative to those tissue or cells without neurodegenerative disease or neuronal damage that do not show an increased level of STAT3 activity.

In certain aspects, the present disclosure provides methods of reducing neuroinflammation or glial alteration in the brain of a subject, comprising administering a therapeutically effective amount of a S1QEL or a S3QEL to a subject in need thereof.

In certain embodiments, following administration of the S1QEL or S3QEL, an inflammatory marker or a glial reactivity marker in the subject's brain is reduced. In certain embodiments, the inflammatory marker or the glial reactivity marker is a tau-related inflammatory marker for example selected from CD52, Itgb2, Irf8, Hmox1, CD83, Ctsb. In certain embodiments, the inflammatory marker or the glial reactivity marker is a pan astrocyte marker selected from Gfap and Vim. In certain embodiments, the inflammatory marker or the glial reactivity marker is an A1 reactive astrocyte marker, for example selected from Ggta1, Gbp2, H2-D1, Serping 1, and H2-T23. In certain embodiments, the inflammatory marker or the glial reactivity marker is an A2 reactive astrocyte marker Emp1. In certain embodiments, the inflammatory marker or the glial reactivity marker is a pan microglia marker, for example selected from CD68 and Aif1. In certain embodiments, the inflammatory marker or the glial reactivity marker is a disease-associated microglia marker selected from Clec7a, Tyrobp, and Trem2.

In certain aspects, the present disclosure provides methods of treating cancer in a subject, comprising administering a therapeutically effective amount of a S1QEL or a S3QEL to a subject in need thereof.

In certain embodiments, the cancer has aberrantly increased STAT3 levels. For example, the cancer may have at least about 1.1, at least about 1.2, at least about 1.3, at least about 1.4, or at least about 1.5 fold increase in total STAT3 relative to a comparative cancer that is not responsive to a S1QEL and/or a S3QEL, e.g., U-87 MG. In some embodiments, the increase in STAT3 is relative to a comparative cancer that does not show an increased level of STAT3, e.g., U-87 MG. In exemplary embodiments, a cancer having sensitivity to an S3QEL had an increased total STAT3 levels as compared to glioblastoma cell line that was not responsive to S3QEL.

In certain embodiments, the cancer has aberrantly active STAT3. For example, the cancer may have at least about 1.1, at least about 1.2, at least about 1.3, at least about 1.4, or at least about 1.5 fold increase in active STAT3 relative to a comparative cancer that is not responsive to a S1QEL and/or a S3QEL, e.g., U-87 MG. In some embodiments, the increase in active STAT3 is relative to a comparative cancer that is not responsive to a S1QEL and/or a S3QEL. In some illustrative embodiments, the increase in active STAT3 is relative to a comparative cancer that does not show an increased level of STAT3 activity, e.g., U-87 MG. In exemplary embodiments, a cancer having sensitivity to an S3QEL had an increased active STAT3 levels as compared to glioblastoma cell line that was not responsive to S3QEL.

In certain embodiments, the cancer is a brain cancer, such as a glial tumor or a non-glial tumor.

In certain embodiments, the cancer is multiple myeloma, human T-cell leukemia virus type 1 (HTLV-I)-dependent leukemia, acute myelogenous leukemia (AML), large granular lymphocyte leukemia (LGL), EBV-related/Burkitt's lymphoma, mycosis fungoides, cutaneous T-cell lymphoma, non-Hodgkins lymphoma (NHL), anaplastic large-cell lymphoma (ALCL), breast cancer, head and neck cancer, ovarian cancer, lung cancer, pancreatic cancer, prostate cancer, skin cancer, medullary thyroid carcinoma, or glioblastoma multiforme.

In certain embodiments, the S1QEL or S3QEL is administered orally, intraperitoneally, or intravenously. In certain preferred embodiments, the S1QEL or S3QEL is administered orally.

In certain embodiments, the S1QEL or S3QEL is active in the brain for at least 2-20 hours. In certain embodiments, the S1QEL or S3QEL is active in the brain for at least 2-10 hours.

In certain embodiments, the S1QEL is selected from the compounds listed below, and pharmaceutically acceptable salts thereof:

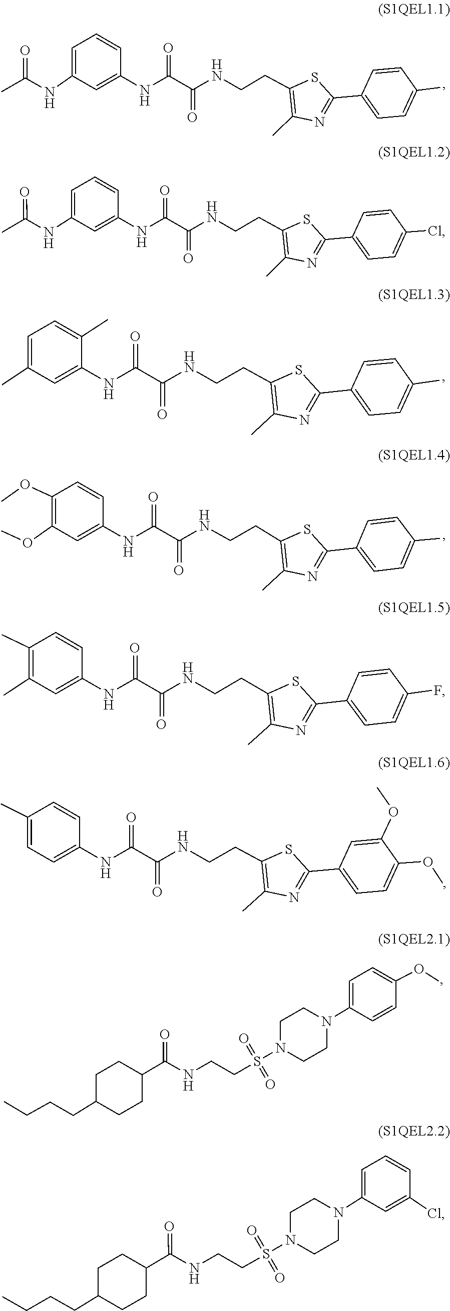

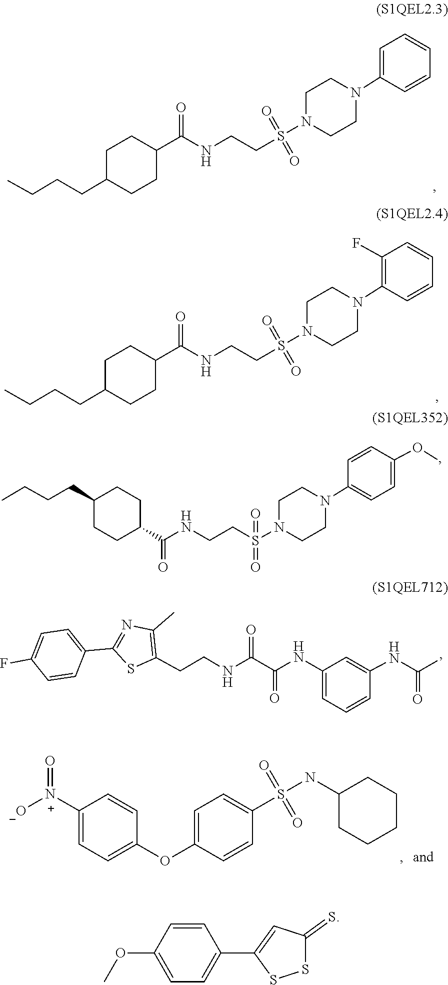

In certain embodiments, the S3QEL is selected from the compounds listed below, and pharmaceutically acceptable salts thereof:

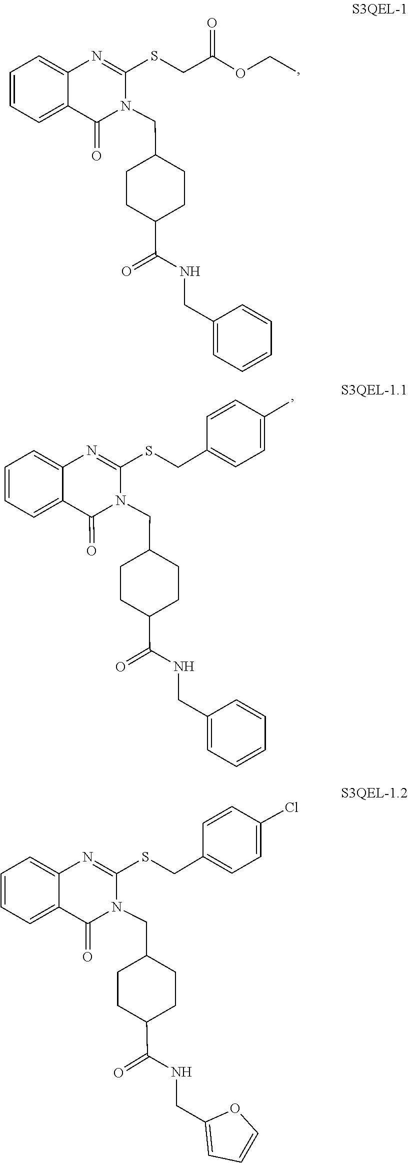

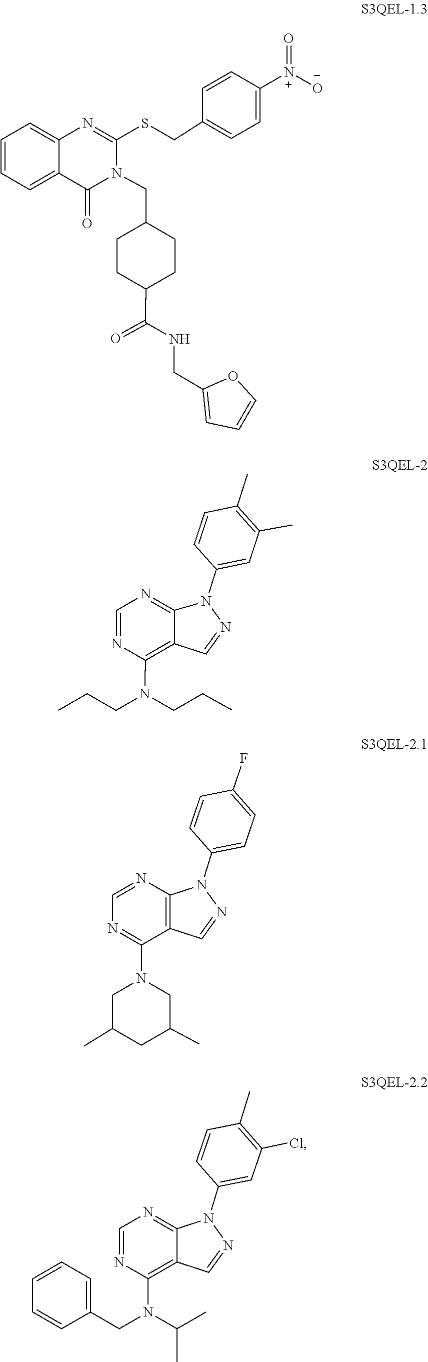

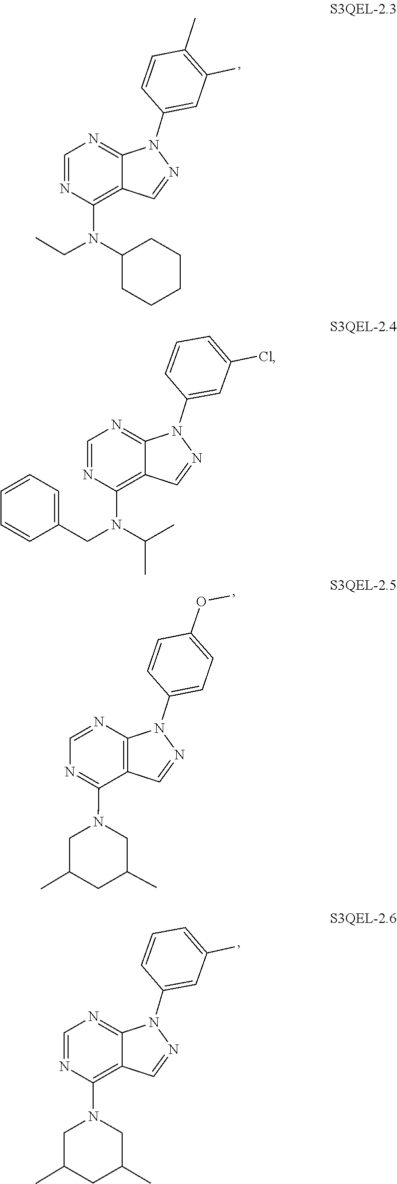

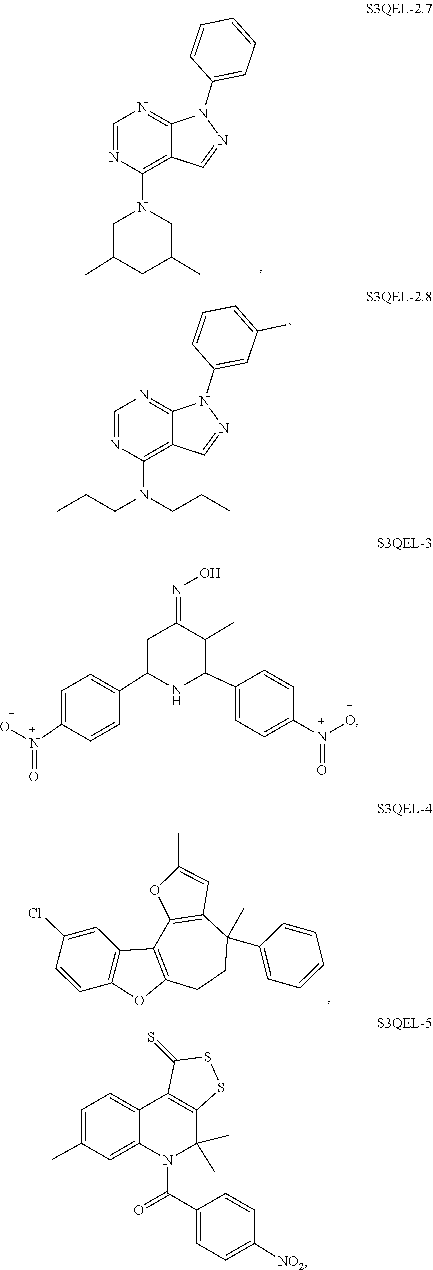

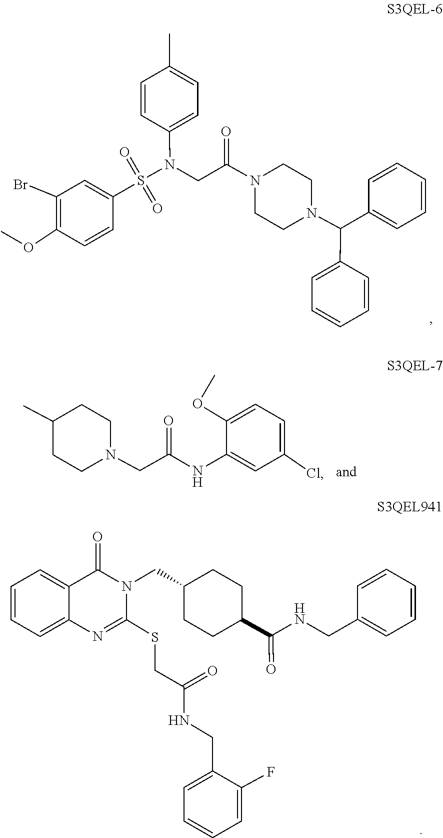 [SMILES: COc1ccc(S(=O)(=O)N(CC(=O)N2CCN(C(c3ccccc3)c3ccccc3)CC2)c2ccc(C)cc2)cc1Br ; COc1ccc(Cl)cc1NC(=O)CN1CCC(C)CC1 ; O=C(CSc1nc2ccccc2c(=O)n1C[C@H]1CC[C@H](C(=O)NCc2ccccc2)CC1)NCc1ccccc1F]

### A. Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic compound like the S1QELs and S3QELs described herein, or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of a neurodegenerative disease includes, for example, reducing the number of instances of the disease in a population of patients receiving a prophylactic treatment relative to an untreated control population, and/or delaying the appearance of detectable symptoms of the disease growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds of the present disclosure. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of the present disclosure are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of the present disclosure for laboratory use, or for subsequent conversion to a pharmaceutically acceptable acid addition salt.

The term “pharmaceutically acceptable basic addition salt” as used herein means any non-toxic organic or inorganic base addition salt of any acid compounds of the present disclosure or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Certain compounds useful in the methods and compositions of this disclosure may have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure. Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce the compound of the present disclosure. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

### Compositions

In another aspect, the present disclosure provides a pharmaceutical composition comprising a compound described herein, optionally admixed with a pharmaceutically acceptable carrier or diluent.

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, the compound of the present disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, cream, lotion or the like. The composition can also be present in a transdermal delivery system, e.g., a skin patch. The composition can also be present in a solution or composition suitable for topical administration.

A pharmaceutically acceptable carrier can contain physiologically acceptable agents that act, for example, to stabilize, increase solubility or to increase the absorption of a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of composition can be a self-emulsifying drug delivery system or a self-microemulsifying drug delivery system. The composition (preparation) also can be a liposome or other polymer matrix, which can have incorporated therein, for example, the compound of the present disclosure. Liposomes, for example, which comprise phospholipids or other lipids, are nontoxic, physiologically acceptable and metabolizable carriers that are relatively simple to make and administer.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, soybean oil (e.g., glycine soja oil), linseed oil (e.g., linum usitatissium seed oil), and eucalyptus oil (e.g., eucalyptus globulus leaf oil); (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, the carrier is selected from polyols (e.g., propylene glycol, butylene glycol, pentylene glycol, hexylene glycol, caprylyl glycol, and glycerin), carbitol, glycol ethers (e.g., ethylene glycol monobutyl ether, propylene glycol monomethyl ether, propylene glycol monobutyl ether, dipropylene glycol and diethylene glycol), alkyl ethers (e.g., diethylene glycol monoethyl ether (ethoxy diglycol) and diethylene glycol monobutyl ether), pyrogen-free water, alcohol (e.g., ethyl alcohol, isopropanol, propanol, butanol, benzyl alcohol, and phenylethyl alcohol).

In some embodiments, the composition is an emulsion. Emulsions include oil-in-water, silicone-in-water, water-in-oil, water-in-silicone, and the like. When formulated as an emulsion, an emulsifier is typically included.

A composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); transdermally (for example as a patch applied to the skin); and topically (for example, as a cream, ointment or spray applied to the skin, or as a product applied to the hair). In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.001 percent to about 99 percent of active ingredient. In some embodiments, this amount will range from about 5 percent to about 70 percent. In some embodiments, this amount will range from about 10 percent to about 30 percent. In some embodiments, this amount will range from about 0.001 percent to about 10 percent by weight of the composition.

Methods of preparing these formulations or compositions include the step of bringing into association an active compound, such compound described herein, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as a wetting agent, an emulsifier, a suspending agent, a sweetener, a flavor, a dye, a fragrance, and a preservative.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, and patches. The active compound may be mixed under sterile conditions with a pharmaceutically acceptable carrier, and with any preservatives, buffers, or solvents that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Examples of suitable aqueous and nonaqueous carriers that may be employed in the compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifiers and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

For use in the methods of this disclosure, active compounds can be given per se or as a composition containing, for example, 0.001 to 99.5% (more preferably, 0.001 to 10%) of active ingredient in combination with a pharmaceutically acceptable carrier.

Actual dosage levels of the active ingredients in the compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the composition required. For example, the physician or veterinarian could start doses of the composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the present disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In some embodiments of the present disclosure, the active compound may be administered two or three times daily. In some embodiments, the active compound will be administered once daily.

The patient receiving this treatment is any animal in need, including primates, in particular humans, and other mammals such as equines, cattle, swine and sheep; and poultry and pets in general.

The invention now being generally described, it will be more readily understood by reference to the following examples which are included merely for purposes of illustration of certain aspects and embodiments of the present invention, and are not intended to limit the invention.

### EXAMPLES

### Example 1. Identification of Selective Suppressors of Complex III ROS

Complex III ROS are implicated in a broad range of pathologies, at least in part due to the ability of complex III to generate high levels of ROS towards the cytosol. However, all pharmacological and genetic tools used to specifically target complex III ROS also depolarize mitochondria, inhibit oxidative phosphorylation, and can affect other targets. Thus, new pharmacological tools with improved selectivity for ROS production are urgently needed.

Any suitable chemical screening and validation tests may be used to identify compounds that are selective suppressors of complex III ROS and do not affect energy metabolism in diverse biological systems. See, e.g., Orr, A. L., et al. Suppressors of superoxide production from mitochondrial complex III. Nat Chem Biol 11, 834-836 (2015).

### Example 2. Complex III ROS Suppressor S3QEL2 Crosses the Blood-Brain Barrier, Engages Oxidative Pathways, and Shows Long-Term Tolerability in FTD-Linked Mouse Models

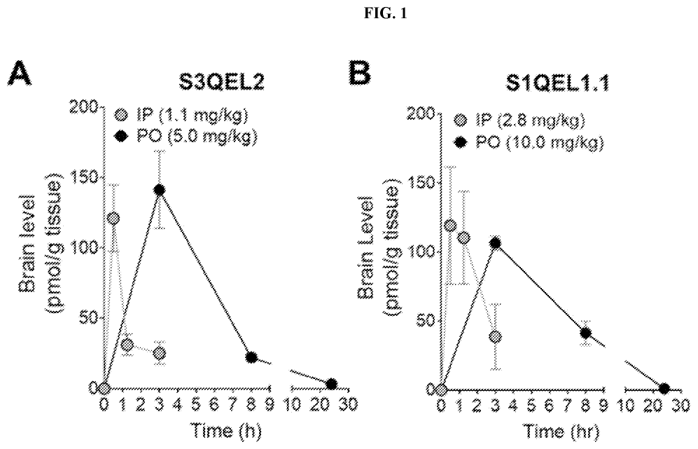

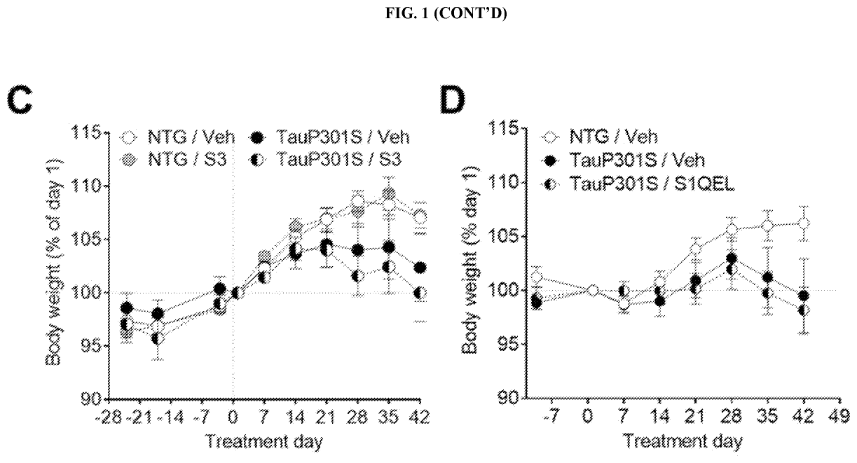

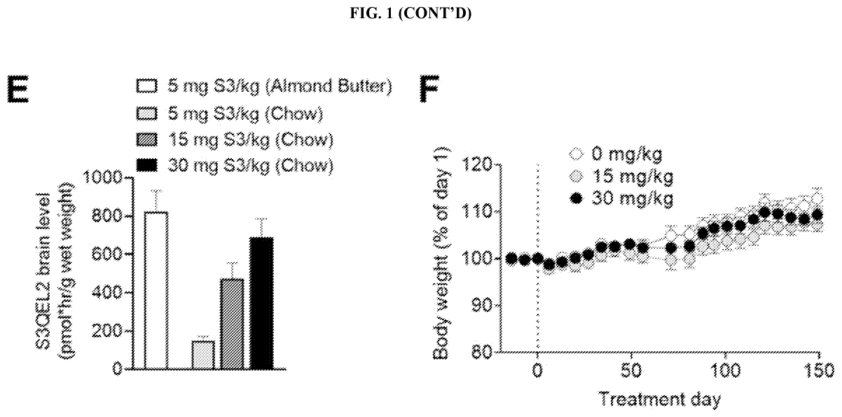

A series of pharmacokinetic and drug tolerance experiments with S3QEL2 in adult wild-type and transgenic hTauP301S mice was first performed. Different administration routes and drug formulations were tested. Due to limited solubility in aqueous solutions, S3QEL2 was administrated at 5 mg/kg/day with DMSO (0.5 ml/kg) mixed in almond butter as the vehicle, which enabled low-stress voluntary consumption by mice for at least six weeks. Importantly, it was found that S3QEL2 and S1QEL1.1 readily crossed the blood-brain barrier after peripheral administration (FIG. 1A-1B). Oral intake resulted in prolonged drug presence in the brain (6-fold or 3.5-fold increase in the area under the curve for S3QEL2 and S1QEL1.1, respectively, as compared to intraperitoneal (i.p.) injection). S3QEL2 or S1QEL1.1 administration did not result in obvious adverse health effects, such as weight loss or altered behavior (FIG. 1C-1D). Chow that was formulated to deliver 0, 5, 15, or 30 mg/kg/day of S3QEL2 was also tested. Chow administration was equally effective in chronically delivering S3QEL2 to the brain of wild-type mice (FIG. 1E), and was tolerated well, even at high doses and long treatment durations in adult mice (FIG. 1F).

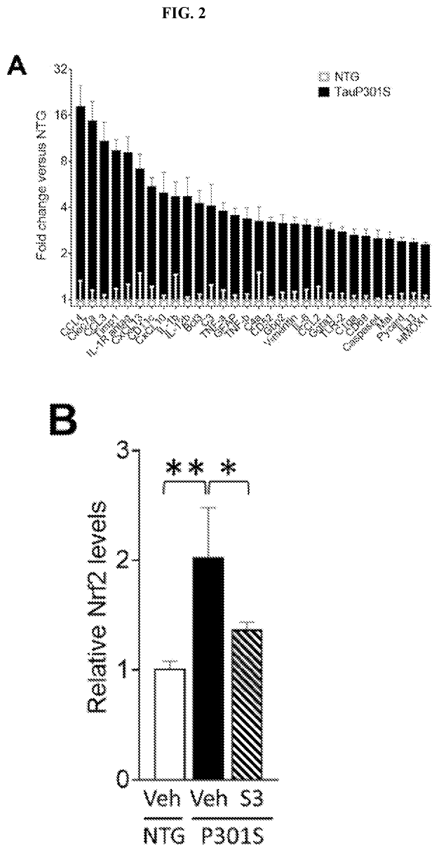

To test the effects of S3QEL in a model of tauopathy, the transgenic hTauP301S line was used, which expresses human 1N4R tau containing the P301S mutation linked to FTDP-17 regulated by the prion protein promoter (JAX 008169). This model exhibits excessive protein oxidation in the brain, possibly due to overproduction of ROS, as well as robust neuropathology, including tau hyperphosphorylation, glial reactivity, neuroinflammation, synaptic loss, behavioral deficits, and early mortality. Indeed, prominent increases in neuroinflammatory gene expression and glial reactivity signatures in the hippocampal formation of hTauP301S mice by 8 months of age was detected (FIG. 2A). These mice also had increased levels of Nrf2, a master regulator of cellular defenses against oxidative stress, and these levels were reduced in hTauP301S mice treated with S3QEL2 for six weeks (FIG. 2B), suggesting that S3QEL2 was effective in modulating oxidative pathways in vivo.

### Example 3. S3QEL2 Reduces Tauopathy, Neuroinflammation, and Early Mortality in Transgenic Mice with FTD-Linked Pathology

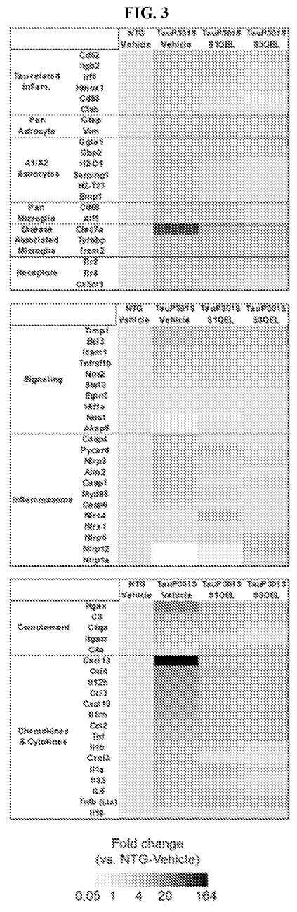

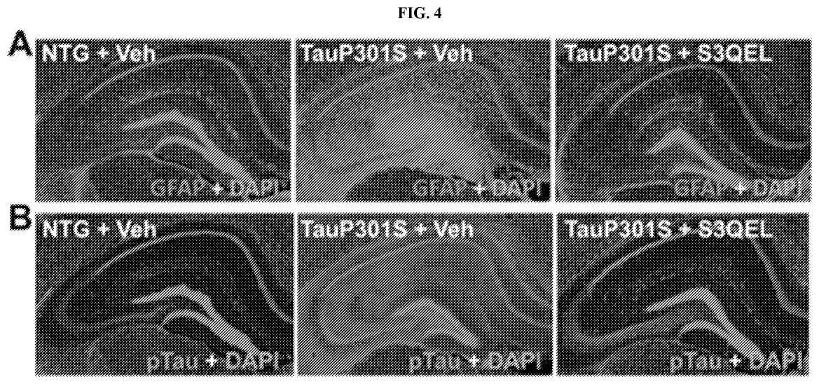

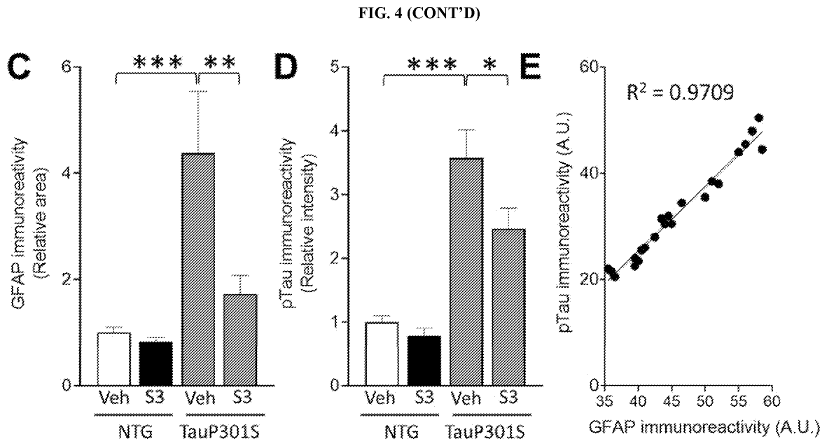

Next, studies were conducted to test if S3QEL2 or SIQEL1.1 altered the expression of genes involved in neuroinflammation and glial reactivity in hTauP301S mice. Targeted transcriptional profiling using a microfluidic-based high-throughput RT-qPCR and a custom-designed panel of over 70 neuroinflammation-related genes revealed that 10-month-old hTauP301S mice treated for six weeks with S3QEL2 or SIQEL1.1 had reduced expression of diverse genes linked to neuroinflammation and glial reactivity in comparison to vehicle-treated controls (FIG. 3).

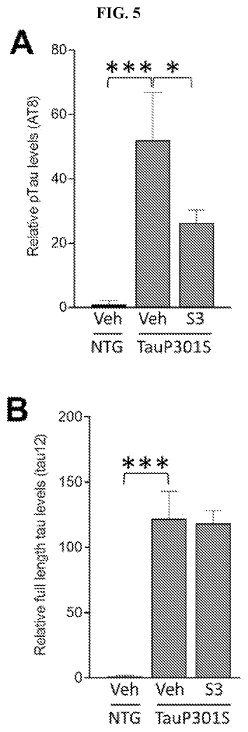

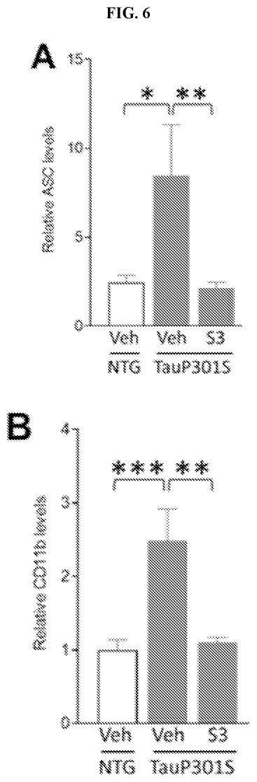

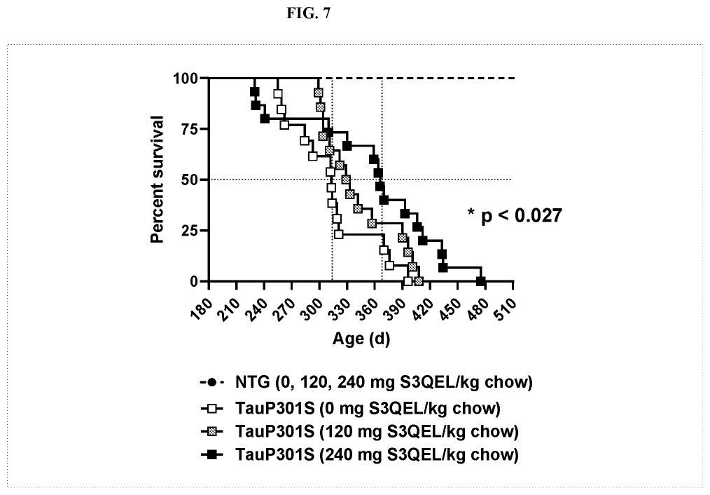

As assessed by immunolabeling, hTauP301S mice treated with S3QEL2 also had reduced levels of glial fibrillary acidic protein (GFAP)-positive astrogliosis and phosphorylated tau (FIG. 7). Western blotting confirmed reduced levels of phosphorylated but not total tau in these mice (FIG. 5). In agreement with the gene expression data, protein levels for neuroimmune-related factors ASC/Pycard and CD11b/Itgam were also reduced in drug-treated hTauP301S mice in comparison to vehicle-treated controls (FIG. 6). These results are consistent with findings that broad immunosuppression reduces tau-associated neuropathology and suggest that complex III ROS may contribute to tauopathy and neuroinflammation.

Survival rates in wild-type and transgenic hTauP301S mice during chronic treatment with control or S3QEL2-containing chow was also monitored. Long-term S3QEL2 administration significantly reduced early mortality associated with tauopathy (FIG. 7).

### Example 4. S3QEL2 Reduces Astrocytic Reactivity and Tauopathy-Linked Neuronal Damage in Isolated Cells

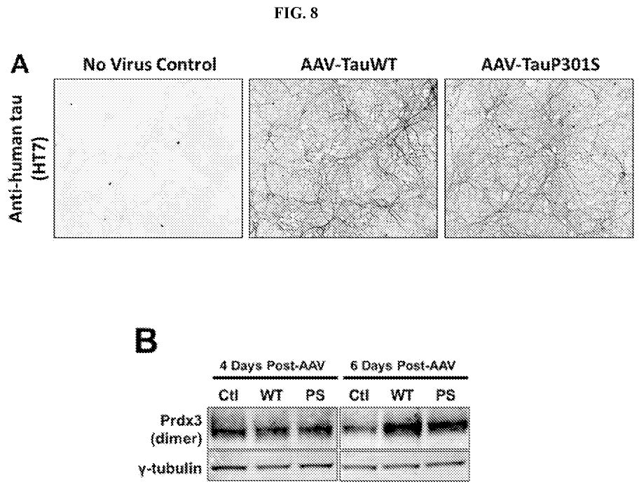

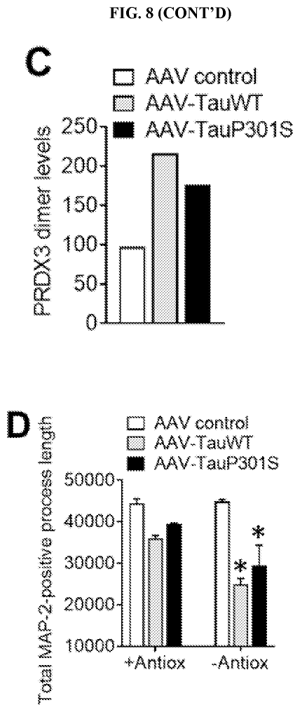

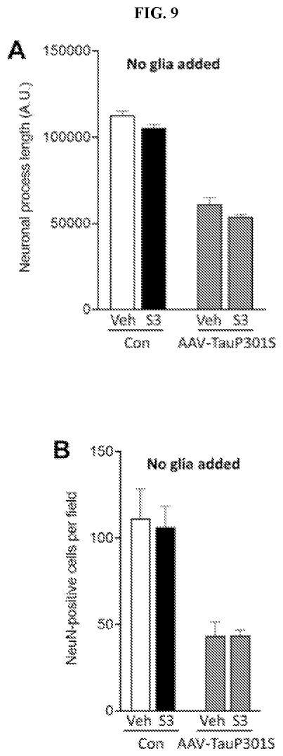

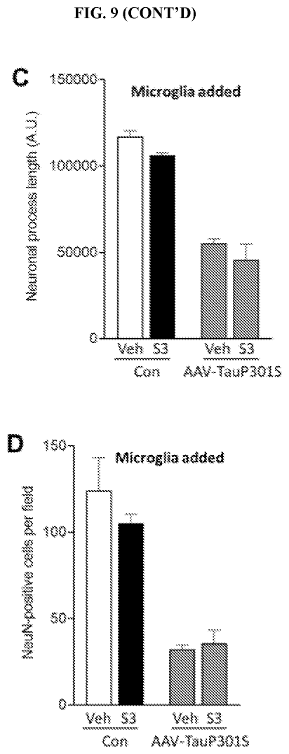

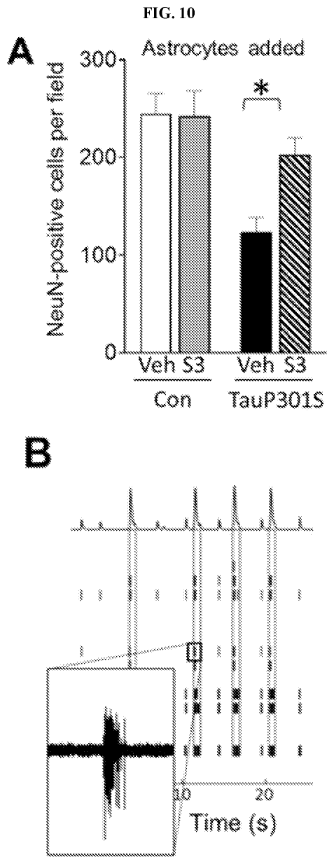

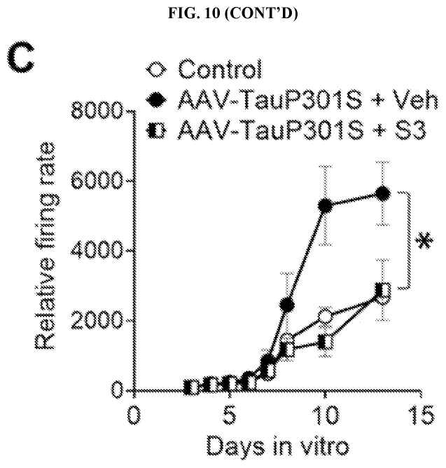

To begin dissecting the mechanisms by which S3QEL2 affects neural cell function and disease-linked processes, studies in primary cultures were conducted. Isolated mouse-derived neurons were transduced with adeno-associated viral vectors (AAVs) encoding wild-type or the P301S mutant form of the human tau protein (FIG. 8A). Tau overexpression increased hyperoxidation-dependent dimerization of peroxiredoxin 3 (PRDX3), a mitochondrial H₂O₂-detoxifying enzyme (FIGS. 8B-8C), suggesting that tau dysregulation induces oxidative stress in neurons. Tau overexpression also reduced microtubule-associated protein-2 (MAP-2)-positive dendritic arbors and neuronal counts, and this degenerative effect was amplified in isolated neurons maintained without antioxidants (FIG. 8D), suggesting that tau-induced neurotoxicity is determined in part by oxidative stress, as suggested previously. Interestingly, S3QEL had no detectable effects on tau-induced neuronal damage in neurons cultured alone (approx. 5% glial cells) or in the presence of microglia (approx. 30% microglia; FIG. 9). However, S3QEL reduced neuronal damage in the presence of astrocytes (approx. 30% astrocytes, FIG. 10A). Tau can also promote aberrant neuronal activity and hypersynchrony. S3QEL reduced aberrant neuronal firing in tau-transduced neurons co-cultured with astrocytes (FIGS. 10B-10C). Together, these data suggest that tau can trigger neuronal damage in a cell-autonomous manner in neurons cultured alone, as well as in a non-cell autonomous manner in neurons cultured with astrocytes. Importantly, these data implicate astrocytic but not neuronal or microglial complex III ROS in tau-related neuronal dysfunction.

Notably, the effects of mitochondrial ROS on glial cells, including astrocytes, are largely unknown. Recent studies suggest that basal mitochondrial ROS production is about 6-10-fold higher in astrocytes compared to neurons and heightened production in disease might affect cell metabolism, neuronal survival, and disease progression. In aging, some of the top downregulated genes in astrocytes are involved in mitochondrial function and antioxidant defenses, suggesting that aging may cause oxidative stress in astrocytes and contribute to aging-related disorders.

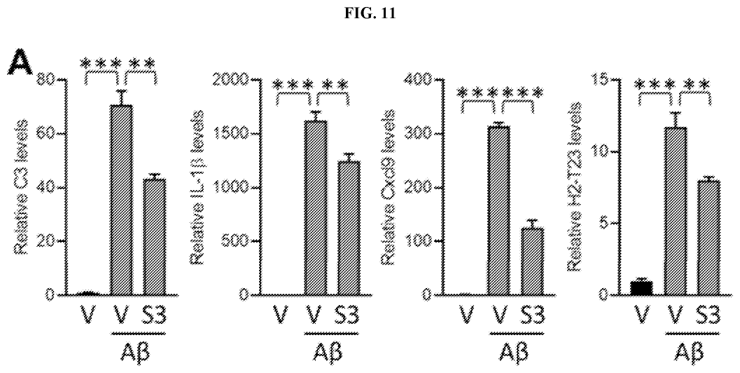

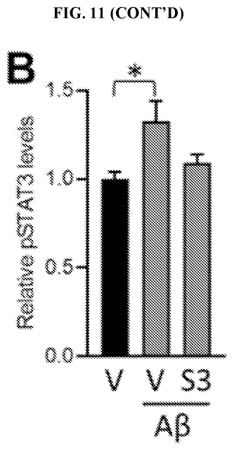

To further assess if S3QELs alter glial functions, the effects of S3QEL2 on isolated astrocytes and microglia was tested. Indeed, S3QEL2 reduced markers of reactivity in isolated astrocytes treated with oligomeric Aβ, which is known to induce astrocytic reactivity (FIG. 11). These markers included genes associated with neurodegeneration and aging, such as complement component C3, which promotes synapse elimination in disease, and phosphorylated STAT3, which is considered a central regulator of astrocytic reactivity.

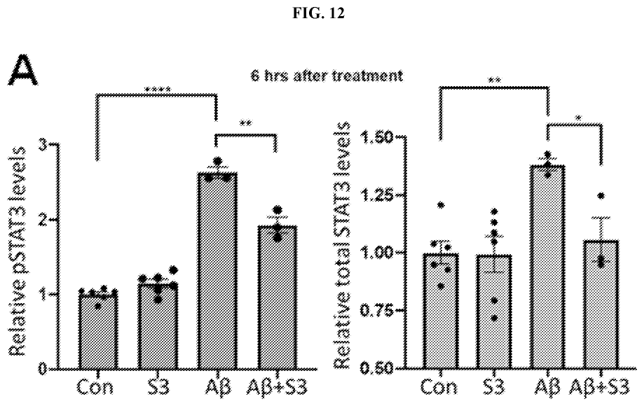

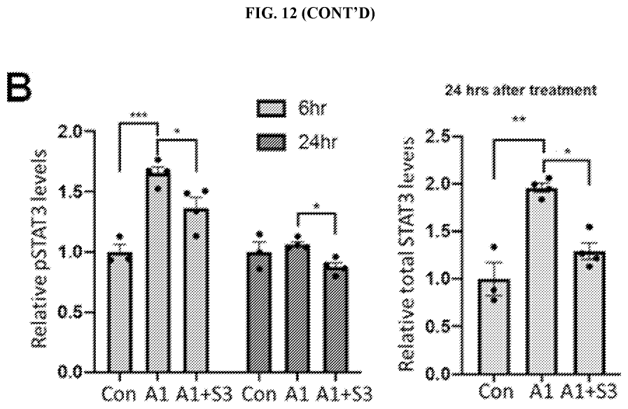

Similar to oligomeric Aβ, A1 cocktail (which is a mixture of TNF-α, IL-1α, and Clq) induced astrocytic reactivity that is associated with synaptic damage and neurodegeneration. S3QEL2 reduced the levels of phosphorylated and total STAT3 in isolated astrocytes treated with oligomeric Aβ or A1 cocktail (FIGS. 11-12), suggesting that S3QEL2 acts to suppress astrocytic reactivity and neuroinflammatory responses.

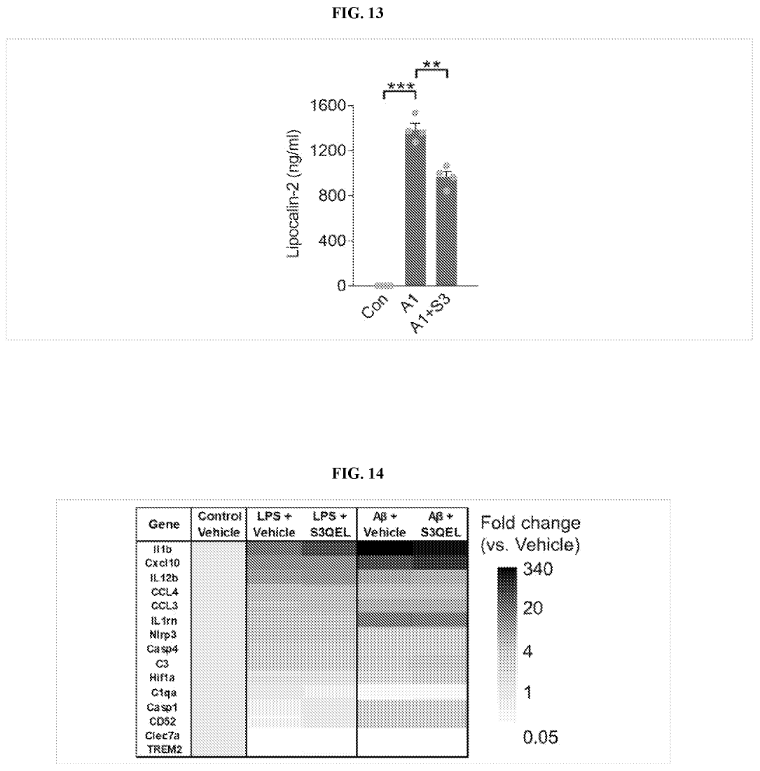

Notably, STAT3 can trigger astrocytic expression and release of neurotoxic factors, including lipocalin-2, which can be damaging to neurons and other cell types, and may promote a vicious cycle of disease-associated cell damage and inflammation. S3QEL2 inhibited the release of lipocalin-2 by isolated astrocytes treated with A1 cocktail (FIG. 13), further suggesting that S3QEL2 reduces pathogenic mechanisms in astrocytes that may contribute to neuroinflammation and neurodegeneration.

### Example 5. The Effects of SELs on STAT3-Dependent Tumorigenesis

As described above, S3QEL2 treatment inhibits STAT3 activation and protein expression in glial cells in the context of disease or inflammation. Notably, STAT3 overactivity contributes to cancers, including brain tumors and their aberrant immune microenvironment. Therefore, studies are conducted to assess the effect of S3QELs on STAT3 signaling that can promote brain tumors, including glial and non-glial tumors.

Briefly, tumorigenesis is assessed in immortalized or primary brain tumor cells, neurospheres, and mouse models implanted with xenografts derived from different types of human brain tumors. Isolated immortalized cells, primary cells, and xenografts of human tumors with gene mutations linked to increased STAT3 signaling are assessed for sensitivity to SELs compared to tumors with mutations that do not increase STAT3 signaling or do not require STAT3 for tumorigenesis. Defined samples are used to identify the sensitivity to SELs of specific types of brain tumors (e.g. specific cell subtypes, genetic mutations, and molecular profiles).

Genetically modified mouse models that develop glioblastomas spontaneously are used to assess whether SELs can prevent tumor occurrence in predisposed or high-risk individuals. In mouse models treated chronically with control/vehicle or SELs, PET ligands are used to track brain tumor evolution and spread over time in a noninvasive manner and obtain a dose-response relationship in vivo.

STAT3 is constitutively activated in diverse types of cancers in addition to brain tumors, and is considered to be a promising molecular target for cancer therapy. Thus, the beneficial effects of SELs on STAT3 activity and tumorigenesis extend to cancer cells from other organs and are not specific to glia or brain cells. In support, aberrant activation of STAT3 has been reported in various human cancer cell lines and tissues, including solid tumors (breast, pancreatic, and prostate cancers) and blood cancers (leukemias and lymphomas). Therefore, studies are conducted to assess the effect of S3QELs on STAT3 signaling in experimental models of non-CNS cancers. For instance, 3T3 fibroblasts stably transformed with the Src oncogene tyrosine kinase and A2058 melanoma cells are examined for STAT3 protein levels and activation, cell proliferation, apoptosis, and p53 levels.

### Example 6. S3QEL2 Reduces Tumor Cell Proliferation

The effect of S3QELs on proliferation of glial tumor cells that have different oncogenic mutations was assessed. In particular, proliferation rates were assessed in three different human immortalized glioblastoma cells lines (A-172, T98G, and U-87 MG). A-172 and U-87 lines have mutations in PTEN, a phosphatase that indirectly regulates PI3K activities, whereas T98G cell line has a mutation in p53, a well-known tumor suppressor gene. STAT3 can affect PI3K signaling and p53 transcription and could thereby promote glioblastoma cell proliferation. Whether S3QELs reduce glioblastoma cell proliferation and aberrant intracellular signaling was tested.

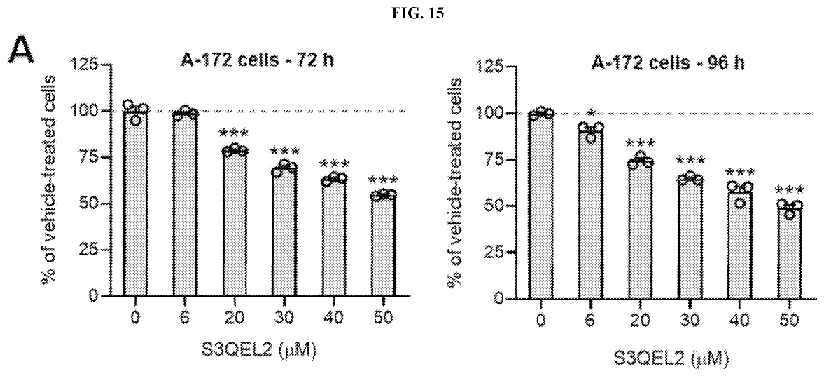

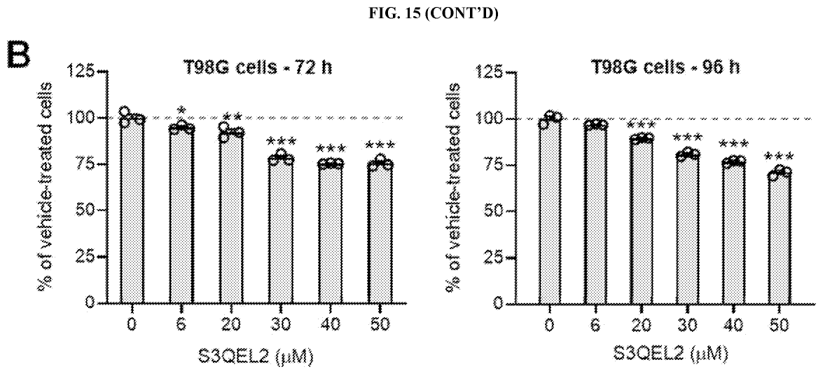

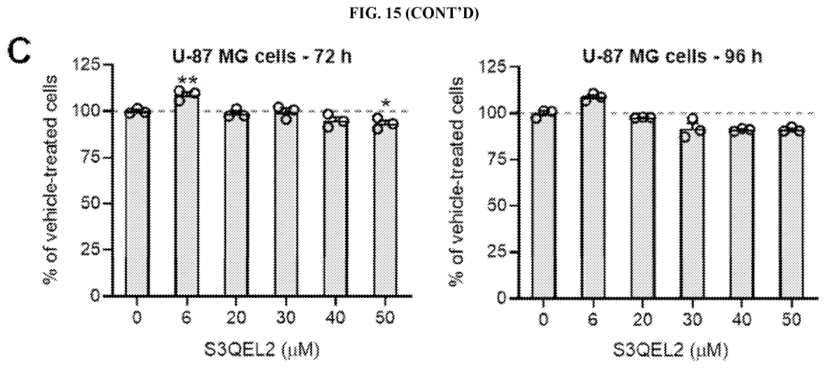

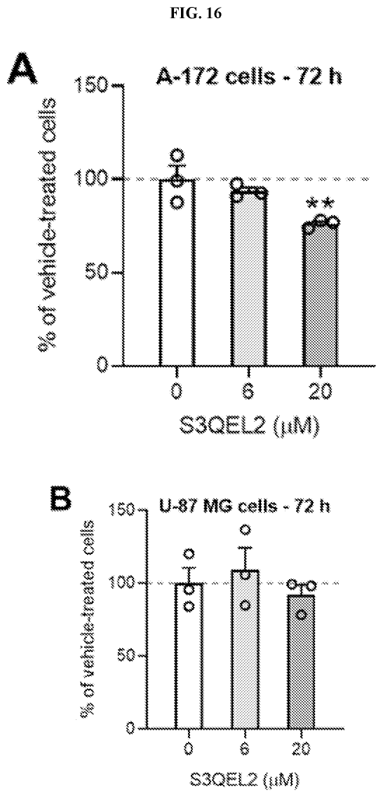

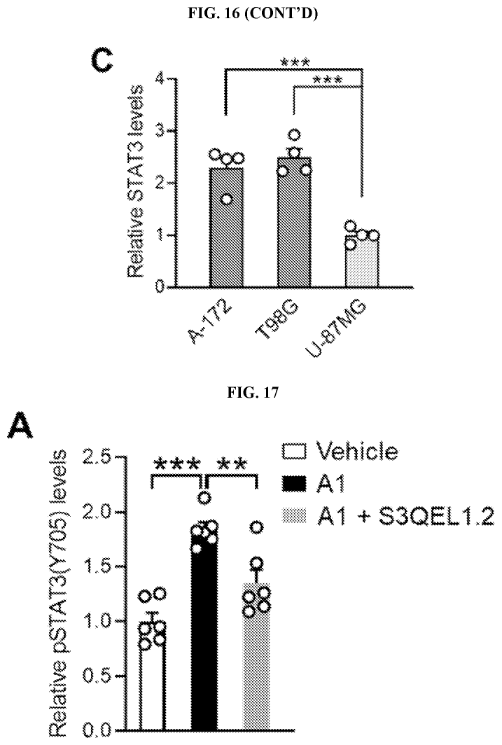

Briefly, two complementary methods were used to assess cell proliferation, including the CellTiter-Glo assay and DAPI staining of cell nuclei. The effects of S3QEL2 treatment on A-172, T98G, and U-87 MG cells were measured at different drug concentrations (0, 6, 20, 30, 40, or 50 μM) and time-points after treatment (72 or 96 hours). S3QEL2 inhibits proliferation of A-172 and T98G glioblastoma cells in a highly dose-dependent manner and these effects are similar at different time-points (FIG. 15A-B). In contrast, S3QEL2 does not have a similar effect on proliferation of U-87 MG cells (FIG. 15C). Consistent with these results, DAPI staining similarly showed that S3QEL2 reduces proliferation of A-172 cells but not U-87 MG cells (FIG. 16A-B). Notably, analysis of basal STAT3 protein levels in the three different glioblastoma cell lines revealed that the two cell lines that showed sensitivity to S3QEL2 treatment also had higher levels of total STAT3 protein as compared to U-87 MG cells that were not sensitive to S3QEL2 (FIG. 16C), suggesting that S3QELs have pronounced beneficial effects in cells with aberrantly increased STAT3 levels.

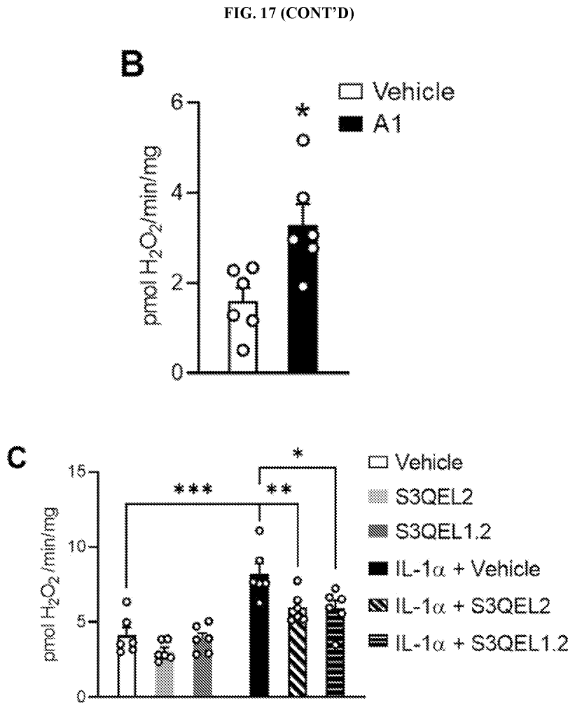

In addition, a different structural analog of S3QEL was studied to see if it had a similar effect on STAT3 activation as S3QEL2. Indeed, like S3QEL2, S3QEL1.2 reduced the levels of phosphorylated STAT3 in isolated astrocytes treated with the A1 cocktail (FIG. 17A). Given that this compound has a different chemical structure, these findings indicate that S3QELs suppress astrocytic reactivity and neuroinflammatory responses through modulation of complex III ROS rather than modulation of other off-target or nonspecific mechanisms.

Given that S3QELs modulate ROS in isolated mitochondria and cell lines, and influence intracellular signaling in astrocytes, whether S3QELs modulate ROS levels in astrocytes at baseline and after exposure to the A1 cocktail, which induces an astrocytic reactivity phenotype associated with neurodegenerative disease, was evaluated. Indeed, 24-hour treatment with the A1 cocktail, which consists of TNF-α, IL-1α, and Clq, increased astrocytic H₂O₂ release as measured using the Amplex UltraRed assay (FIG. 17B). Given that the A1 cocktail is a mixture of three different immune-related factors, whether one of the three factors is sufficient to induce astrocytic ROS production was evaluated. Indeed, 24-hour treatment with IL-1α alone was sufficient to increase astrocytic H₂O₂ levels (FIG. 17C). Moreover, two different structural analogs of S3QEL, S3QEL2 and S3QEL1.2, had minimal effects on baseline H₂O₂ levels, but suppressed the increases in astrocytic H₂O₂ levels induced by IL-1α (FIG. 17C). Together, these findings support the conclusion that S3QELs inhibit astrocytic ROS production associated with aberrant intracellular signaling and neurological disease. Therefore, these types of suppressors of electron leak represent methods of treating neurodegenerative disorders and STAT3-linked cancers.

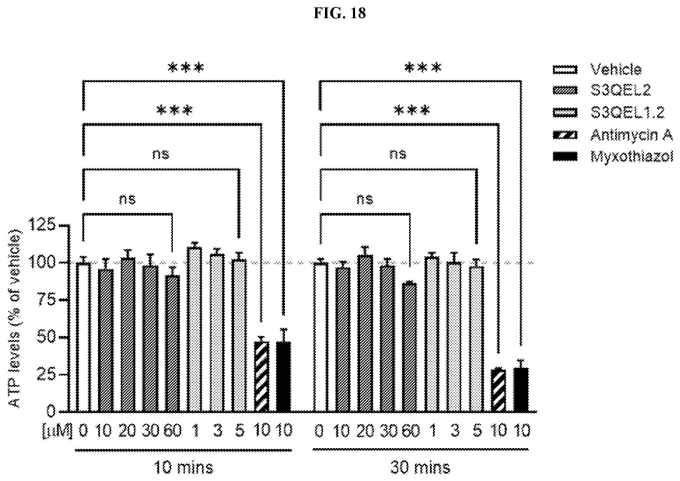

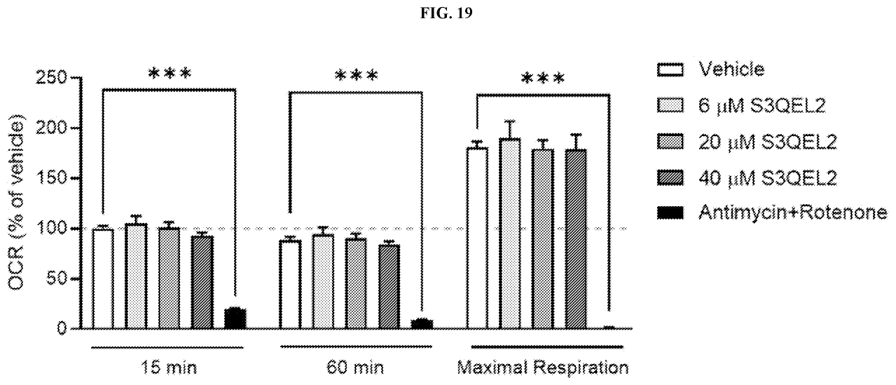

Finally, to further confirm that S3QELs are selective suppressors of complex III ROS and do not affect energy metabolism in astrocytes, the effects of S3QELs on astrocytic ATP production and oxygen consumption rates (OCR) were tested. S3QELs did not alter these readouts of mitochondrial function, whereas the positive controls (mitochondrial respiration inhibitors antimycin A, myxothiazol, and rotenone) were able to greatly reduce ATP and OCR levels (FIG. 18-19).

Interestingly, STAT3 is constitutively enhanced in diverse types of cancers and is therefore a promising molecular target for cancer therapy. The effects of S3QELs on STAT3 and tumor cell proliferation likely extend to cancers in other organs and are not specific to glia or brain cells. In support, aberrant activation of STAT3 has been reported in various human cancer cell lines and tissues, including solid tumors (breast, pancreatic, and prostate cancers) and blood cancers (leukemias and lymphomas).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or preventing a neurodegenerative disease or neuronal damage, comprising administering a therapeutically effective amount of a S1QEL or a S3QEL to a subject in need thereof.

2. The method of claim 1, wherein following administration of the S1QEL or S3QEL, an inflammatory marker or a glial reactivity marker in the subject's brain is reduced.

3. The method of claim 1 or 2, wherein the neurodegenerative disease or neuronal damage is Alzheimer's disease, Parkinson's disease, amyotrophic lateral sclerosis, argyrophilic grain disease, chronic traumatic encephalopathy, corticobasal degeneration, dementia, progressive supranuclear palsy, frontotemporal dementia, cerebral amyloid angiopathy, chronic pain, Creutzfeldt-Jakob disease, depression, Huntington's disease, spinal cord injury, traumatic brain injury, HIV-associated neurodegeneration, intracerebral hemorrhage, multiple sclerosis, stroke, vascular dementia, medullary thyroid carcinoma, or glioblastoma multiforme.

4. The method of claim 1 or 2, wherein the neurodegenerative disease or neuronal damage is a tauopathy.

5. The method of claim 4, wherein the tauopathy is dementia, Alzheimer's disease, Parkinson's disease, progressive supranuclear palsy, corticobasal degeneration, argyrophilic grain disease, or chronic traumatic encephalopathy.

6. The method of claim 1 or 2, wherein the neurodegenerative disease or neuronal damage is dementia, such as frontotemporal dementia or amyotrophic lateral sclerosis.

7. The method of claim 1 or 2, wherein the neurodegenerative disease or neuronal damage is Alzheimer's disease.

8. The method of claim 1 or 2, wherein the neurodegenerative disease or neuronal damage is Parkinson's disease.

9. A method of reducing neuroinflammation or glial alteration in the brain of a subject, comprising administering a therapeutically effective amount of a S1QEL or a S3QEL to a subject in need thereof.

10. The method of claim 9, wherein following administration of the S1QEL or S3QEL, an inflammatory marker or a glial reactivity marker in the subject's brain is reduced.

11. The method of claim 10, wherein the inflammatory marker or the glial reactivity marker is a tau-related inflammatory marker selected from CD52, Itgb2, Irf8, Hmox1, CD83, and Ctsb.

12. The method of claim 10, wherein the inflammatory marker or the glial reactivity marker is a pan astrocyte marker selected from Gfap and Vim.

13. The method of claim 10, wherein the inflammatory marker or the glial reactivity marker is an A1 reactive astrocyte marker selected from Ggta1, Gbp2, H2-D1, Serping1, and H2-T23.

14. The method of claim 10, wherein the inflammatory marker or the glial reactivity marker is an A2 reactive astrocyte marker Emp1.

15. The method of claim 10, wherein the inflammatory marker or the glial reactivity marker is a pan microglia marker selected from CD68 and Aif1.

16. The method of claim 10, wherein the inflammatory marker or the glial reactivity marker is a disease-associated microglia marker selected from Clec7a, Tyrobp, and Trem2.

17. A method of treating a cancer, comprising administering a therapeutically effective amount of a S1QEL or a S3QEL to a subject in need thereof.

18. The method claim 17, further comprising determining a STAT3 level of the cancer prior to administering the therapeutically effective amount of the S1QEL or S3QEL.

19. The method of claim 17 or 18, wherein the cancer has aberrantly increased STAT3 levels.

20. The method of claim 17, 18, or 19, wherein the cancer has aberrantly active STAT3.

21. The method of any one of claims 17-20, wherein the cancer is a brain cancer, such as a glial tumor or a non-glial tumor.

22. The method of any one of claims 17-20, wherein the cancer is multiple myeloma, human T-cell leukemia virus type 1 (HTLV-I)-dependent leukemia, acute myelogenous leukemia (AML), large granular lymphocyte leukemia (LGL), EBV-related/Burkitt's lymphoma, mycosis fungoides, cutaneous T-cell lymphoma, non-Hodgkins lymphoma (NHL), anaplastic large-cell lymphoma (ALCL), breast cancer, head and neck cancer, ovarian cancer, lung cancer, pancreatic cancer, prostate cancer, skin cancer, medullary thyroid carcinoma, or glioblastoma multiforme.

23. The method of any one of claims 1-22, wherein the S1QEL or S3QEL is administered orally, intraperitoneally, or intravenously.

24. The method of any one of claims 1-22, wherein the S1QEL or S3QEL is administered orally.

25. The method of any one of claims 1-24, wherein the S1QEL or S3QEL is active in the brain for at least 2-20 hours.

26. The method of any one of claims 1-24, wherein the S1QEL or S3QEL is active in the brain for at least 2-10 hours.

27. The method of any one of claims 1-26, wherein the method comprises administering an S1QEL selected from:

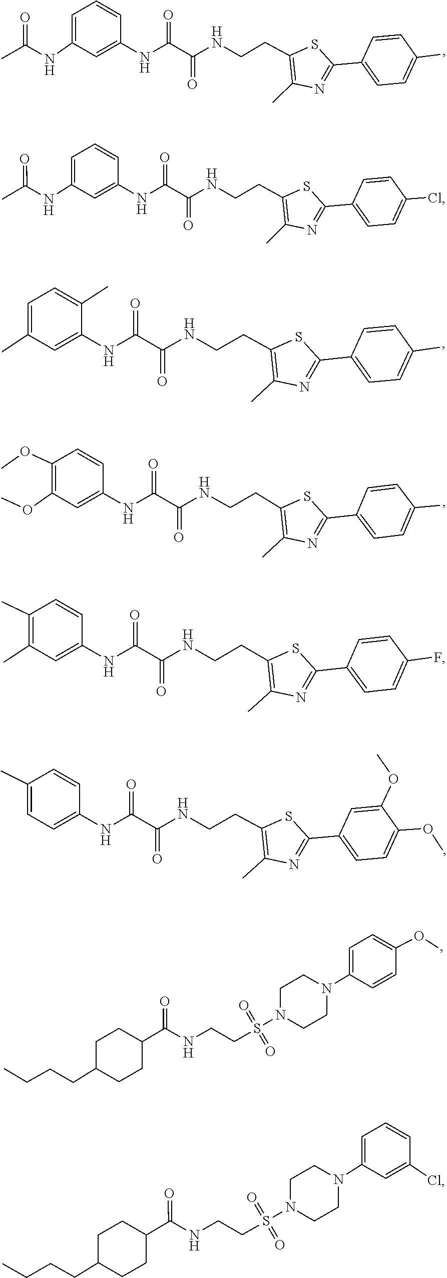

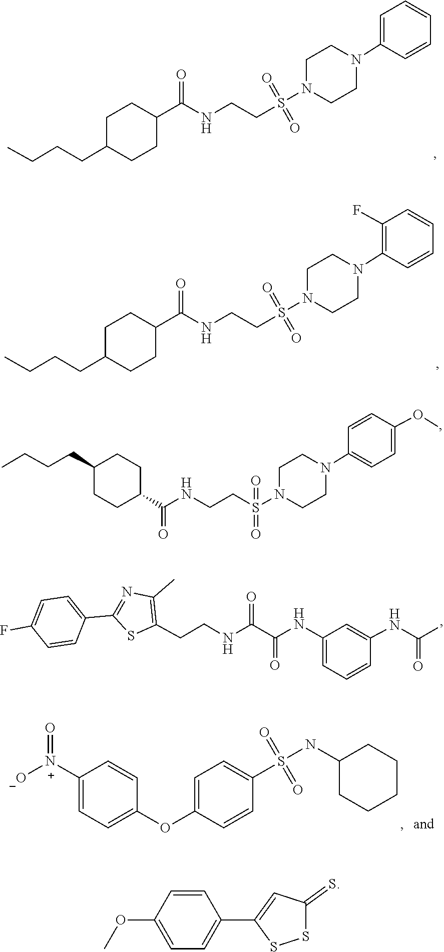

28. The method of any one of claims 1-26, wherein the method comprises administering a S3QEL selected from: 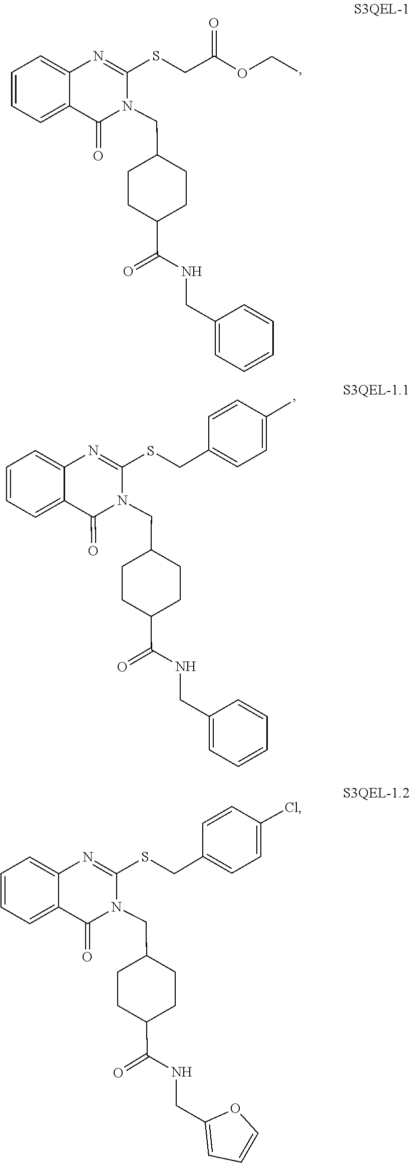 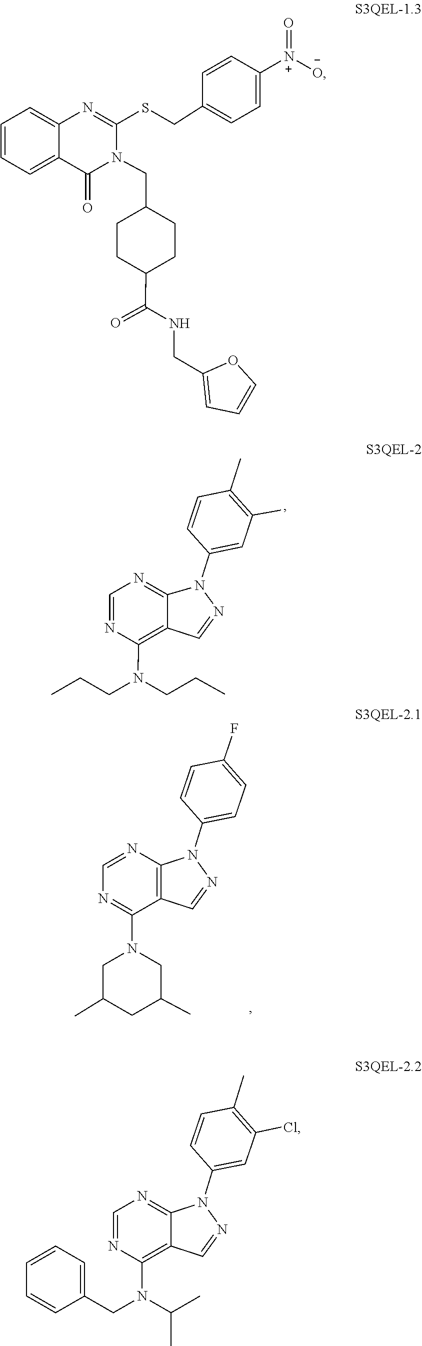 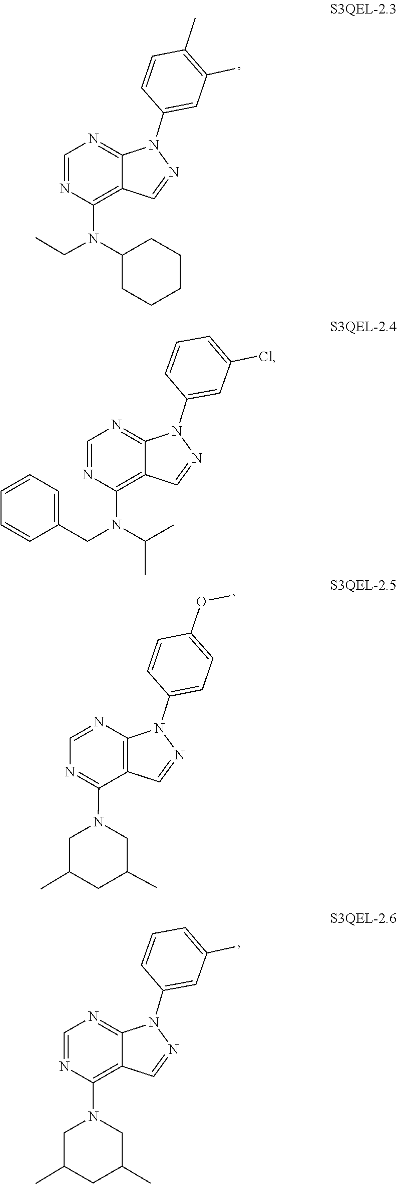 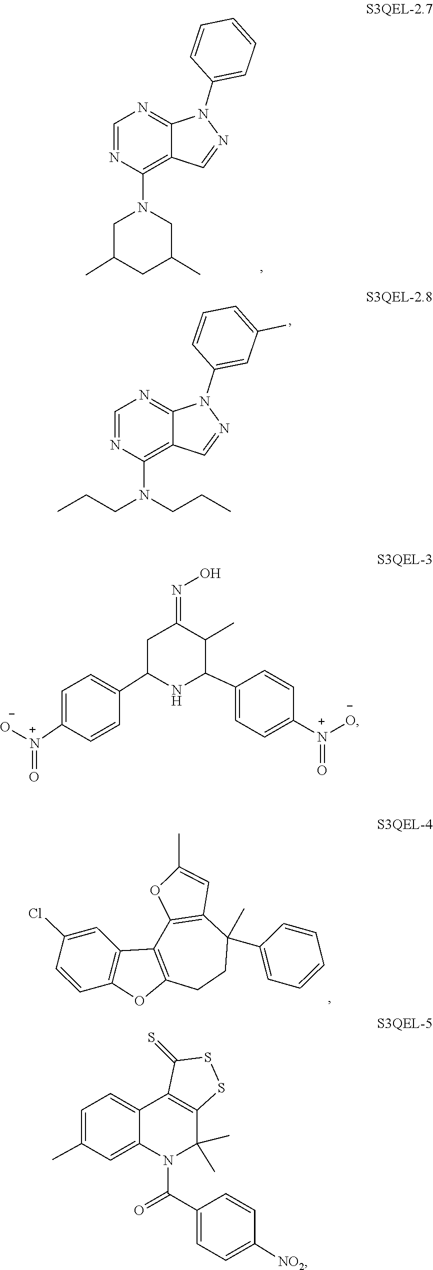 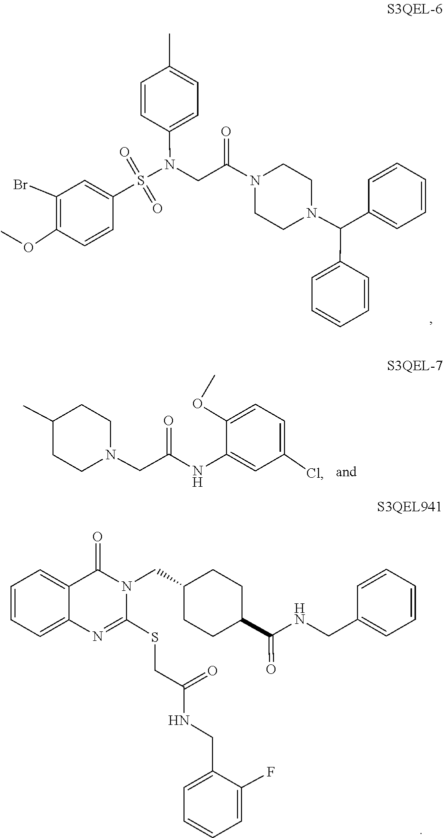 [SMILES: COc1ccc(S(=O)(=O)N(CC(=O)N2CCN(C(c3ccccc3)c3ccccc3)CC2)c2ccc(C)cc2)cc1Br ; COc1ccc(Cl)cc1NC(=O)CN1CCC(C)CC1 ; O=C(CSc1nc2ccccc2c(=O)n1C[C@H]1CC[C@H](C(=O)NCc2ccccc2)CC1)NCc1ccccc1F]
